(19) United States
(12) Patent Application Publication
(10) Pub. No.: US 2024/0145101 A1
(43) Pub. Date: May 2, 2024

(54) SYSTEM AND METHOD FOR IDENTIFYING INFECTION RISK

(71) Applicant: AIRFIELD & INDUSTRIAL EQUIPMENT LTD., Campbellford (CA)

(72) Inventors: Stephen Lyle MCKEOWN, Campbellford (CA); Ty SHATTUCK, Burlington (CA); Paul Edward CUDMORE, Castleton (CA)

(21) Appl. No.: 18/568,611

(22) PCT Filed: Jun. 8, 2022
(86) PCT No.: PCT/CA2022/050916

§ 371 Date: Dec. 8, 2023

Related U.S. Application Data
(60) Provisional application No. 63/209,223, filed on Jun. 10, 2021.

(51) Int. Cl. G16H 50/80

(52) U.S. Cl. CPC G16H 50/80

(57) ABSTRACT

Systems and methods for identifying infection risks are disclosed. A system for identifying infection risk includes: one or more sensors associated with each of a plurality of physical assets for generating a signal when two or more physical assets of the plurality of physical assets are in a predetermined proximity; and a server configured to receive the signal from the one or more sensors, and to identify an infection status of each of the two or more physical assets, all governed by a business model as defined by the inventors.

### FIELD

The present application relates to holistic biological threats prevention, in particular, to systems and methods for identifying infection risk across a wide range of jurisdictions through a flexible and adaptable business and technological solution.

### BACKGROUND

It is important to timely and accurately identify emerging biological threat outbreaks to reduce the risk of outbreaks or pandemics.

### SUMMARY

The present application provides systems, methods and processes for integrating, analyzing and reporting upon real-time, real-world data related to biological threats across dispersed geographies. While outbreaks of contagious biological threats are inevitable, pandemics are preventable with swift and data-driven interventions. The methods of integration may be achieved through use of networked IoT devices at a physical asset level, aggregated using containers defined as digital twins of the real-world.

The systems and methods of the present application may predict areas of infection risk and suggest applicable mitigating actions. The systems and methods may provide individuals, companies, organizations, agencies and governments the data necessary to identify emerging outbreaks and take appropriate actions to minimize the risk of outbreaks becoming pandemics while allowing normal activities to take place to the extent possible, in a given, protected area/region/jurisdiction.

### BRIEF DESCRIPTION OF THE DRAWINGS

Reference will now be made, by way of example, to the accompanying drawings which show example embodiments of the present application, and in which:

FIG. 1 is a schematic diagram of a system for identifying infection risk, according to an example embodiment;

FIG. 2A is a diagram illustrating exemplary transactions of containers;

FIG. 2B is a diagram illustrating a blockchain for recording transactions of the containers in FIG. 2A;

FIG. 3 is a diagram illustrating an exemplary status machine of the containers in FIG. 2A;

FIG. 4 is a diagram illustrating exemplary intersections of the containers in FIG. 2A;

FIG. 5 is a diagram illustrating exemplary cleaning transactions of the containers in FIG. 2A;

FIG. 6 is a diagram illustrating exemplary test transactions of the containers in FIG. 2A;

FIG. 7 is a diagram illustrating an exemplary model of the containers in FIG. 2A;

FIG. 8 is a diagram illustrating an exemplary blockchain structure, according to an embodiment; and

FIG. 9 is a diagram illustrating an exemplary neural network for the analytic platform in FIG. 7.

Similar reference numerals may have been used in different figures to denote similar components.

### DESCRIPTION OF EXAMPLE EMBODIMENTS

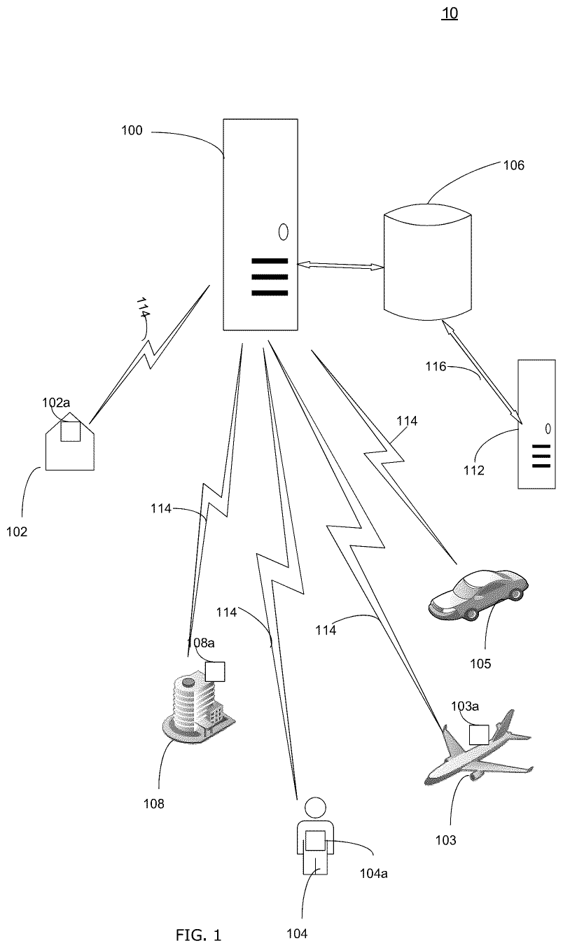

FIG. 1 illustrates a system 10 for identifying infection risks, according to an example embodiment. The system 10 may include a server 100, a database 106, a plurality of physical assets, including houses 102, aircrafts 103, individuals 104, and vehicles 105, buildings 108, etc.

The server 100 may associate each physical asset with one or more sensors, such as IoT sensors. For example, houses 102 are associated with the sensors 102a, aircrafts 103 are associated with the sensors 103a, individuals 104 are associated with the sensors 104a, vehicles 105 are associated with the sensors 105a, and buildings 108 are associated with the sensors 108a. Each sensor is uniquely associated with a physical asset in the server 100. The sensors may be IoT chips. The sensors can transmit signal containing measurement data to the server 100 via a communication link 114. The communication links 114 can be provided by a wireless or wired communications network. In some examples, sensors can transmit signal containing measurement data to a data reader in a close proximity of the sensors via a near field communications (NFC), such as a Bluetooth™, and infrared. The data reader may then transmit the data from the sensors to the server 100 via communication links 114. If a physical asset is movable, the physical asset may be associated with a GPS or GPS-enabled sensor that is uniquely associated with the physical asset for recording the location of the physical asset.

A sensor may be configured to detect the presence of other sensors carried by other physical assets in the proximity of the sensor. For example, a sensors may detect other sensors within a distance of 2 meters. The sensors in a proximity may exchange identity information of the sensors with each other. By exchanging identity information with each other, each sensor has identity information of other sensors in the proximity. Each sensor 102a-105a and 108a may send a signal to report, via a communication link 114, to the server 100 the exchanged information, including the time of identify information exchanged, and the identity information of one or more sensors with which the sensor has exchanged identity information. Each sensor may also report to the server 100 the location of the physical asset, for example, using the GPS coordinates of the physical asset.

In some examples, use of the sensors in system 10 may be governed by a dedicated entity, such as a technical advisory board of the public health authority, and the use of the sensors is adapted to the needs identified by a specific region, area, or jurisdiction, to best monitor or control infection risk.

As well, the server 100 may create a digital twin for each of the physical asset. For example, the server 100 may create a digital or virtual asset for each physical asset, and associate the physical asset with its corresponding physical asset. For example, when one or more sensors collect data from their associated physical asset, the sensor data can be used to update a digital twin copy of the asset's status in real time in the server 100 as reflected in the corresponding digital asset. The digital twin is an up-to-date and accurate copy of the physical asset's properties and status in server 100, including position, status and motion, as reported by the sensors 102a-105a and 108a.

In the present application, a digital twin may be used for monitoring the status of its associated physical assets 102-105 and 108. Sensory data can be combined with GPS data to monitor the movement and location of a physical asset real time.

The server 100 can be a cloud server. The system 10 in this case is a cloud platform, which allows monitoring of the physical assets globally.

In some examples, digital twins may be divided into digital containers, where each digital container delineates a specific physical asset. Each individual 104, vehicle 105, house 102, building 108, aircraft 103, etc., may be defined as a container with the attributes of the container defined by the digital twin, which are linked to the server 100 via sensors. In the system 10, each physical asset may be considered as a container.

When each physical asset encounters other physical assets, for example a mobile physical asset such as an individual 104, a vehicle 105, or an aircraft 103 moves around to encounter a mobile physical asset or a stationary physical asset, such as a house 102 or building 108, the container of the physical asset intersects with the containers of other physical assets

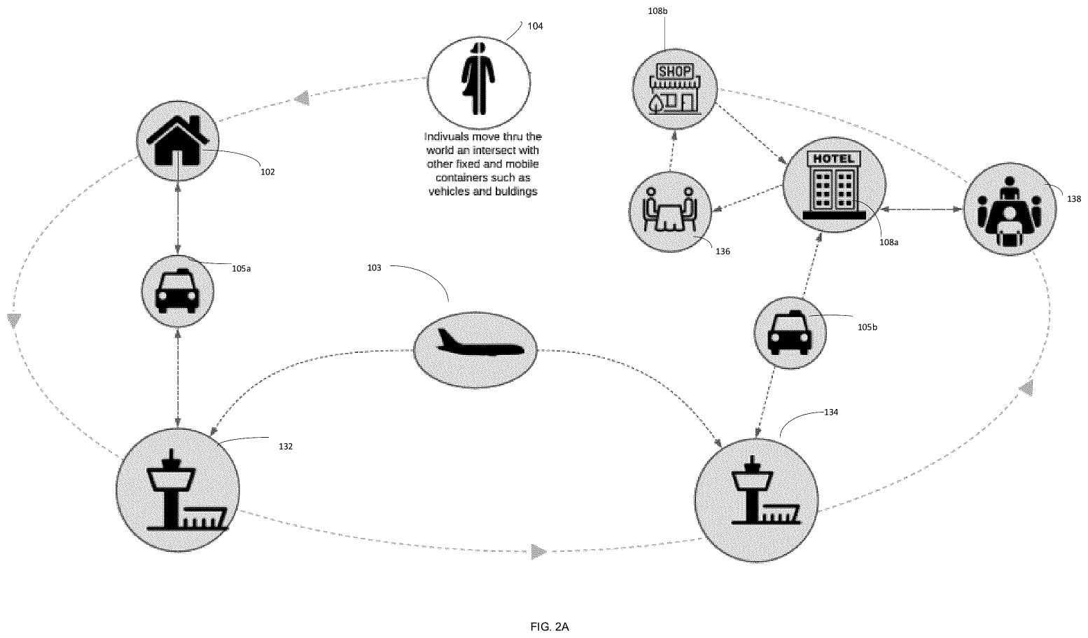

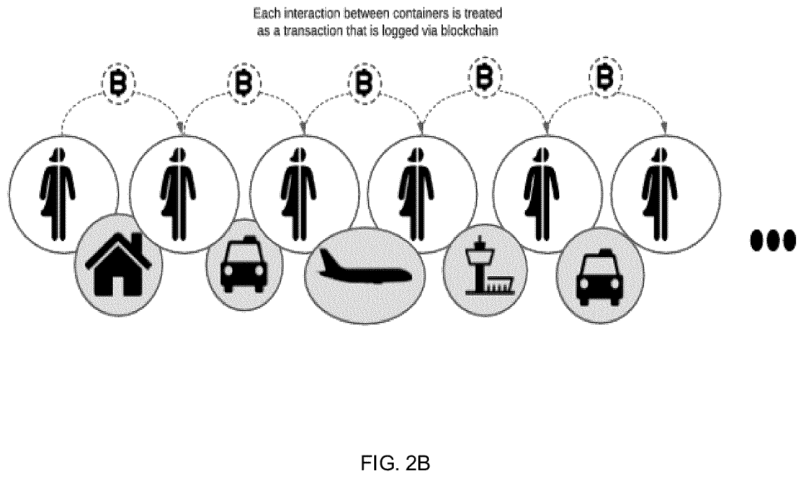

In the example of FIG. 2, a traveler 104 may travel from a house 102 via a vehicle 105a, such as an Uber car, to the airport 132. The traveler 102 travels to a new city via an airplane 103 to a second airport 134, and takes a second vehicle 105b, such as an Uber car, to a hotel 108a. In the city, the traveler 102 may have a conference with other individuals 138, and/or have dinner with other individuals 136, and go shopping at a shop 108b. The example in FIG. 2 involves the following containers:
1. the traveler 104, other individuals 136, 138, and the Uber drivers (person containers);
2. the traveler's house 102, hotel 108a, shop 108b, the airports 132 and 134 (building containers); and
3. the vehicle 105a and 105b, airplane 103 (vehicle containers);

When traveler 104, vehicle 105, aircraft 103, or other mobile physical assets move around in the real-world, their digital twin containers intersect. Each intersection of a container is treated as a transaction. For example, when the traveler 104 enters the Uber vehicle 105a, the container of the traveler 102 intersects with the container of the Uber vehicle 105a and the container of the Uber driver. An intersection occurs when two or more physical assets or containers are within a predetermined proximity, such as, but not limited to, within 2 meters between two physical assets. The sensors associated with the physical assets send signals to the server 100 to report the intersection to the server 100. In response to the report of intersection, the server 100 generates a transaction for the containers involved in the transaction.

As illustrated in FIG. 2B, the server 100 may record each transaction of containers in a block of a blockchain to provide a secure and anonymous log of physical assets interacting in the real-world, as will be described in greater detail below. For example, the transactions between the container of the traveler 102 and each of the containers of the house 102, the vehicle 105a and 105b, the airport 132 may be recorded in a block of a blockchain.

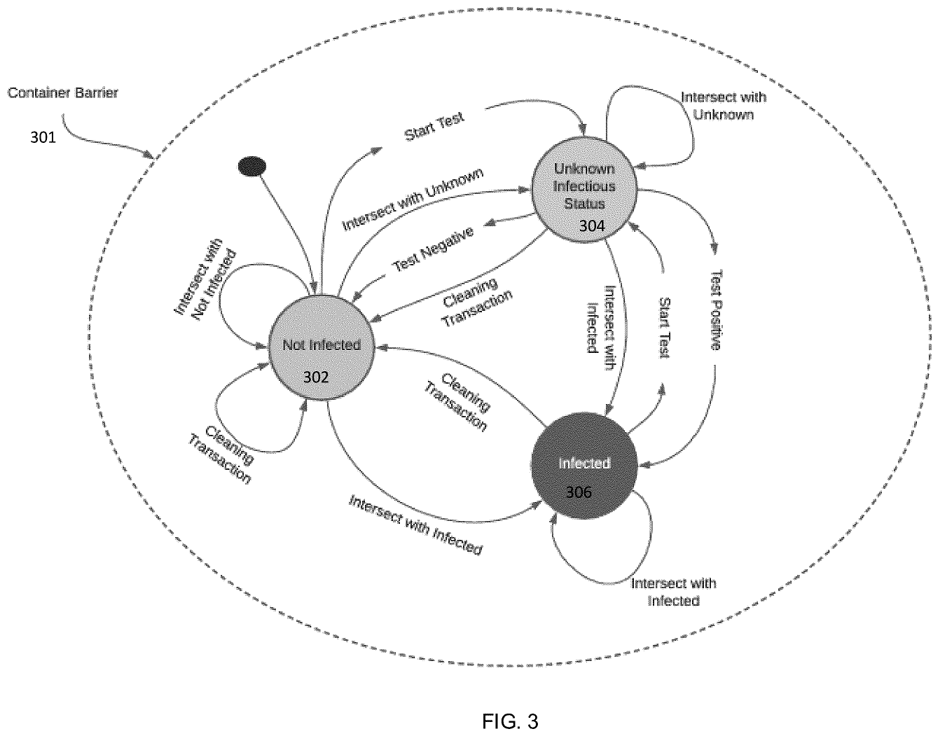

In some examples, a building container may be configured to include a plurality of sensors for continually monitoring existence of pathogens. For example, the sensors may be deployed at the entrances or exits of the building or on various floors, in various rooms of the building. If any one of those sensors indicates that a pathogen is present, namely that a container with an infected status to be described below, the sensor may transmit a signal with sensory data to the server 100 where the digital twin of the physical asset associated with the sensor is virtually stored and that status of that container will be changed according to the status machine in FIG. 3. Similarly, mobile containers such as individuals and vehicles may have GPS-enabled sensors that will report locations real-time or at predetermined periods. As such, the sensors may provide information about the time/period and the location of where two or more containers intersect.

In some examples, the geographic area approximated by a container may be indicated, for example highlighted, by the server 100 in system 10 as an area of risk by information collected and analyzed by earth, aerial or space-based sensors 102a-105a and/or 108a. for example, the server 100 may monitor changes in human behavior of a traveler 104 that are indicative of biological threat outbreaks. Containers within these geographic areas of risk can be assigned lower risk threshold triggers.

Containers may be divided into two types: (1) object containers which represent physical objects (people, vehicles, buildings, etc.) in the real-world, and (2) action containers which represent actions (cleaning action, testing action) that occur within the real world. As illustrated in the status machine in FIG. 3, an object container 301 may have one of three status: (1) not infected 302, (2) unknown infectious status 304, and (3) infected 306.

A container is not infected if in the case of a non-human object container, the container is cleaned according to a defined standard or have a transaction with a cleaning container, or in case of a human container, the container is tested with a negative result according to a defined standard. A container is in an unknown infectious status if there is a transaction with an unknown container status, or if the physical asset is under testing. The test result may be sent to the server 100 via a third party server 112. A container is infected if there is a transaction with another container with an infected status 306, or the sensor associate with the container detects contamination. In some examples, the sensors 102a or 108a may detect infection of a building or room container 102 or 108 through air sniffers, for example Kontrol™ air sniffers, or through testing of persons within the room or building container 102 or 108, such as via rapid COVID testing. The sensors 102a or 108a may transmit air monitoring results to the server 100, or the test results may be uploaded to the third party server 112 by a third party, such as a public health authority. In response to determining that a physical asset is infected, the sever 100 may send an infection risk alert to a person associated with the physical asset or health authority. In some examples, the server 100 may identify when and where an outbreaks are occurring, or likely to occur, and generate recommendations of possible mitigations with respect to relevant containers, regions, and time to prevent outbreaks or pandemic from occurring.

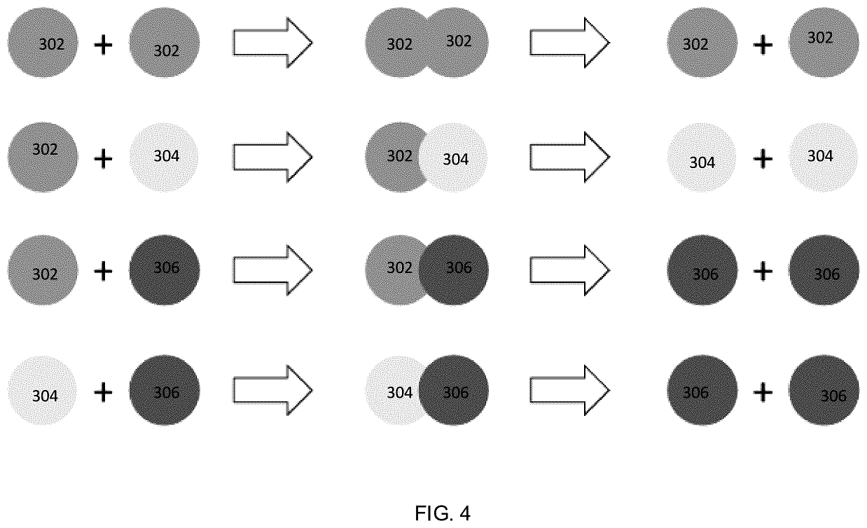

The status of an object container may change by interacting with other object containers and actions containers. The change of status may be reported by the third party server 112 or determined by the server 100 based on the types of transactions. As illustrated in FIG. 4, when two containers having status 302 intersect, the status remain the same to be uninfected 302. When two containers having a status 302 and a status 304 intersect, the status of both containers become 304. When two containers having a status 302 and a status 306 intersect, the status of both contains become 306. When two containers having a status 304 and a status 304 intersect, the status of both containers become 306.

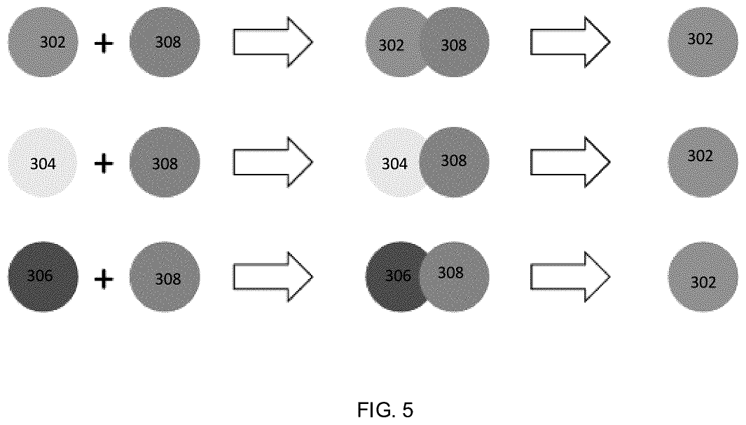

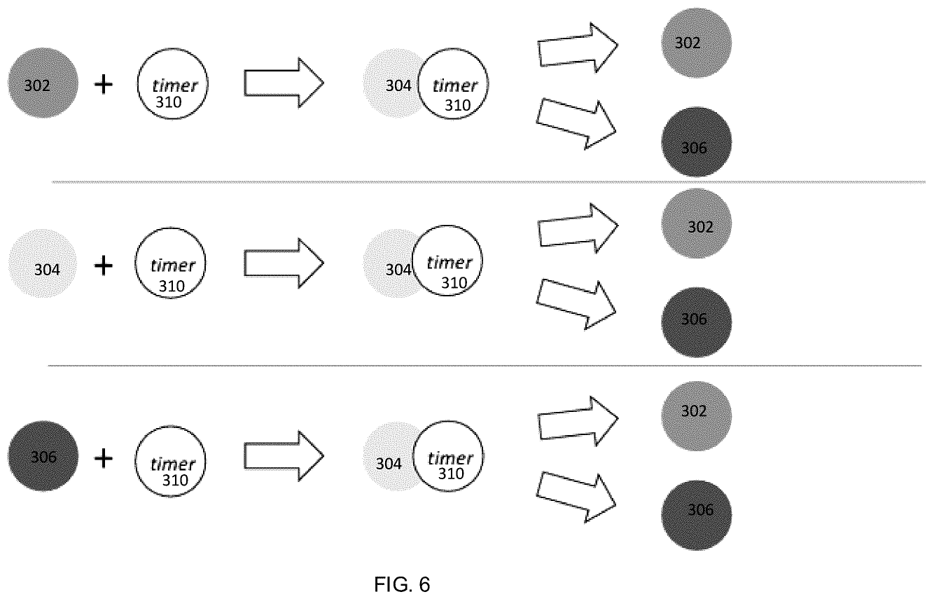

Once an infected transaction occurs, a container's status changes to infected status 306 until an uninfected transaction occurs. As illustrated in FIG. 5, when a container having a status 302 intersects with a cleaning or disinfecting container with status 308, which is an action container, by disinfecting the container, the container becomes uninfected 302. When a containers having a status 304 intersects with a cleaning or disinfecting container with status 308 by disinfecting the container, the container becomes uninfected 302. When a containers having a status 306 intersects with a cleaning or disinfecting container with status 308 by disinfecting the container, the container becomes uninfected 302.

Where a transaction results in a container identified as infected or unknown status, the server 10 may recommend the testing or disinfection actions necessary to address the situation. When containers with status 302, 304, and 306 may change status when they intersect with a Sensor/Test plus timer 310, which is an action container. As illustrated in FIG. 6, containers with status 302, 304, and 306 may intersect with test timer container 310, the tested status include uninfected 302, or infected 306.

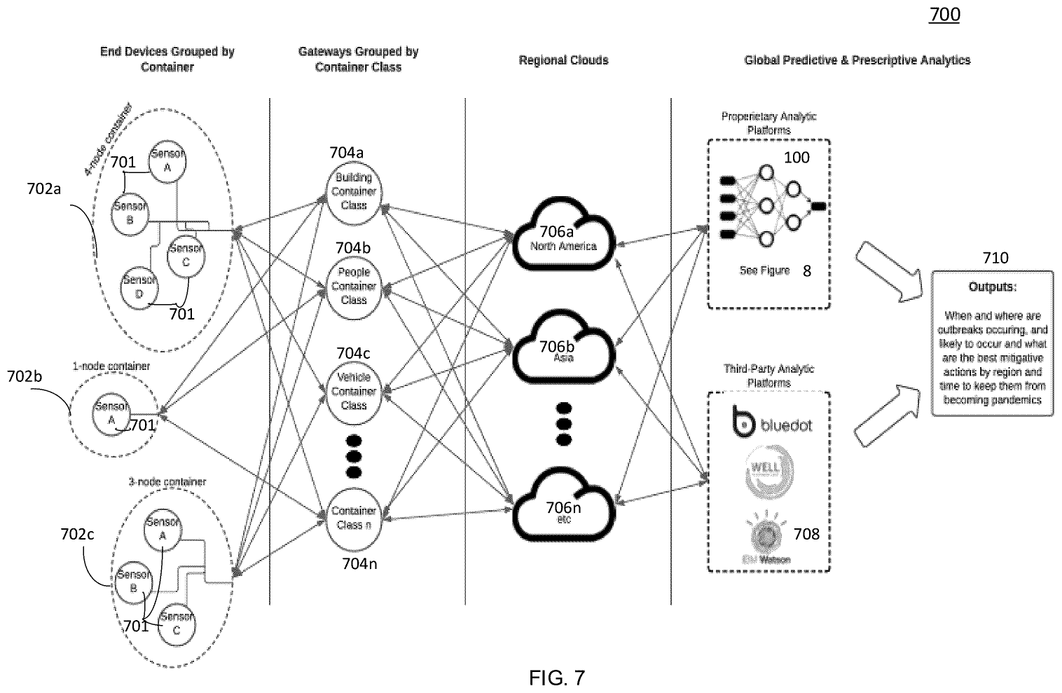

As illustrated in FIG. 7, a container 702a, 702b, or 702c may include one or more sensors (n-nodes) 701, each sensor 701 is associated with a container 702a, 702b, or 702c. The containers 702a, 702b, or 702c may be grouped by container class, such as a building container class 704a if the physical asset associating a sensor 701 in system 10 is a building, a people container class 704b if the physical asset associating a sensor 701 in system 10 is an individual, a vehicle container class 704c if the physical asset associating a sensor 701 in system 10 is a vehicle, or other container class 704n if the physical asset associating a sensor 701 in system 10 is other specific class defined by the server 100. Optionally, the information collected by the sensor 701 may protect anonymity of an individual in the system 10, for example, by associating the information collected by the sensor 701 with the container identification rather than personal information of the individual, such as name and address of the individual.

Different classes of containers 702a-702c may be grouped by geographical regions, such as North America if the containers are located in North America, Asia if the if the containers 702a-702c are located in Asia, or other appropriate regions 706n, which may be defined in server 100. The status of containers 702a-702c may be monitored and processed at an analytic platforms provided in server 100, and/or at a third-party analytic platforms 708 such as Bluedot™.

Grouping different classes of containers in different regions allows the server 100 to identify infection risks when trends are emerging. For example, the server 100 may determine that containers at a specific location x, y or in a specific region today are intersecting ‘more often’ with infected containers than yesterday. The server 100 may in turn generate a heat map or warning.

As well, server 100 is configured to store the sensory data in the database 106, process the received sensory date, identify infection risk, for example, using artificial intelligence, generate and send alert to relevant physical assets and stakeholders.

When a container intersects with a cleaning or disinfecting container with status 308 by disinfecting the container, the intersections are reported to the server 100 by sensors of relevant containers, including action containers and object containers. In some examples, the test transaction and the disinfection transaction may be reported to the server 100 by a third party server 112 in association with the disinfection container. The server 102 may determine the new status of a container based on the status change mechanisms described above in view of FIGS. 3, 4, 5 and 6.

Based on the status of the containers 702a-702c, and the transactions between the containers 702a-702c, based on the status changes described in FIGS. 3-6 above, the analytic platform provided by server 100 is configured to generate outputs, which include identifying when and where outbreaks are occurring, or likely to occur, and generating recommendations of possible mitigations with respect to relevant containers, regions, and time to prevent outbreaks or pandemic from occurring.

In some examples, sensory data received from the sensors 701 associated with containers 702a-702c may be securely stored in one or more blockchains. For example, the server 100 may create a blockchain to record the transactions between two or more containers 702a-702c. The blockchain may be stored in the database 106. The blockchain includes a plurality blocks. The blockchain makes the records stored therein safe against modifications.

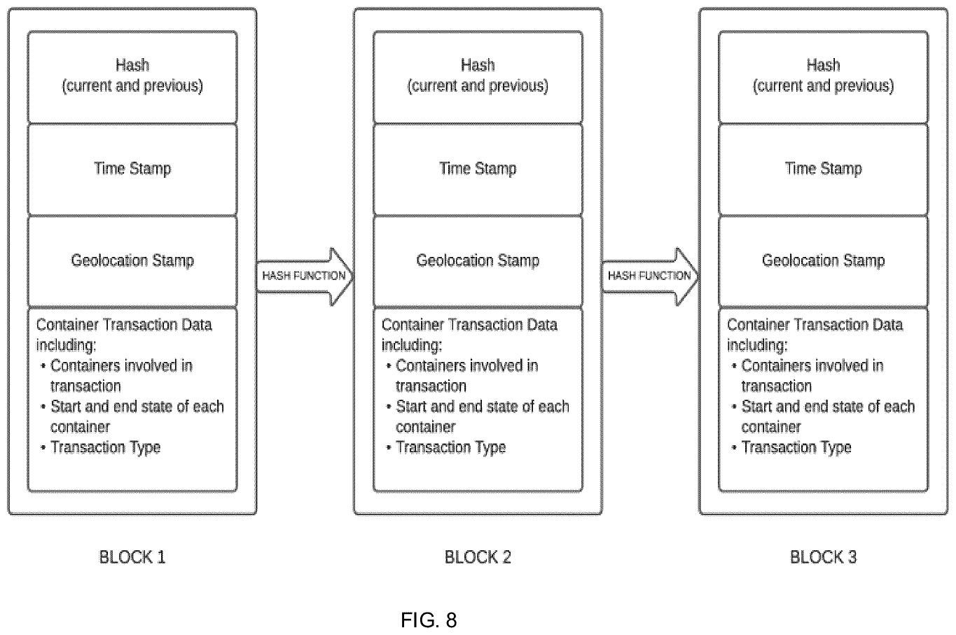

For example, a transaction may be stored, by the server 120, into a block of a blockchain. The structure of a blockchain may include a timestamp to indicate the time of the transaction, a hash value for the previous block, a hash value of current block, identification of the sensor reporting the transaction, the status of the physical asset associated with the sensor ID, the identification of the containers associated with the sensor, geolocation location of the containers, start and end states of each container, types of transaction, and other applicable information. An example of a blockchain structure is illustrated in FIG. 8. In an example, the Hash value of the current block may be calculated by applying hash function over the timestamp to indicate the time of the transaction, the hash value for the previous block, identification of the sensor reporting the transaction, the status of the physical asset associated with the sensor ID, the identification of the containers associated with the sensor, and geolocation location of the containers. The hash function may be MD5, RIPEMD, SHA-1, and SHA-256. A one-way hash function maps data of arbitrary size (called the “message”) to a bit string of a fixed size (the “hash value”, “hash”, or “message digest”). Each of hash for the previous block [n−1], and hash for current block [n] generates a message digest. The blockchain information can be later used to enquire about each transaction of a specific container.

In some examples, all container transactions and all predicted status may include a time component. Since infections are always detected after the time of infection, the system 10 may use the blockchain log of actual container transaction to backtrack in time to determine a container intersected with all other containers at a selected period. Blockchain provides an auditable, secure and anonymous way to record actual container transactions.

The database 106 can also store infection information of a physical asset, the status of a physical asset, the infection history of a physical asset, the locations of a physical asset in real-time or at various time periods, the information provided in the signals from sensors, the associations between the sensors with corresponding physical assets, and between the physical assets and the corresponding digital twins, the blockchain, etc. The database 106 can be integrated in the server 100, or provided in a separate server, such as a server of a public health authority.

In some examples, the database 106 can communicate with the remote server 112 of public health authority, such as via a communication link 116, to receive updated infection diagnosis results of a physical asset from the remoted server 112.

### AI

In some examples, the server 100 may also use a machine learning model to identify infection risk of a physical asset. The machine learning model can be implemented by an artificial neural network (also referred to simply as a “neural network”) running on a computing platform such as server 100. As illustrated in FIG. 8, a neural network 800 may be used in server 100 to process the received sensory date, identify infection risk, generate alert, and send the alert to relevant physical assets or public health authorities.

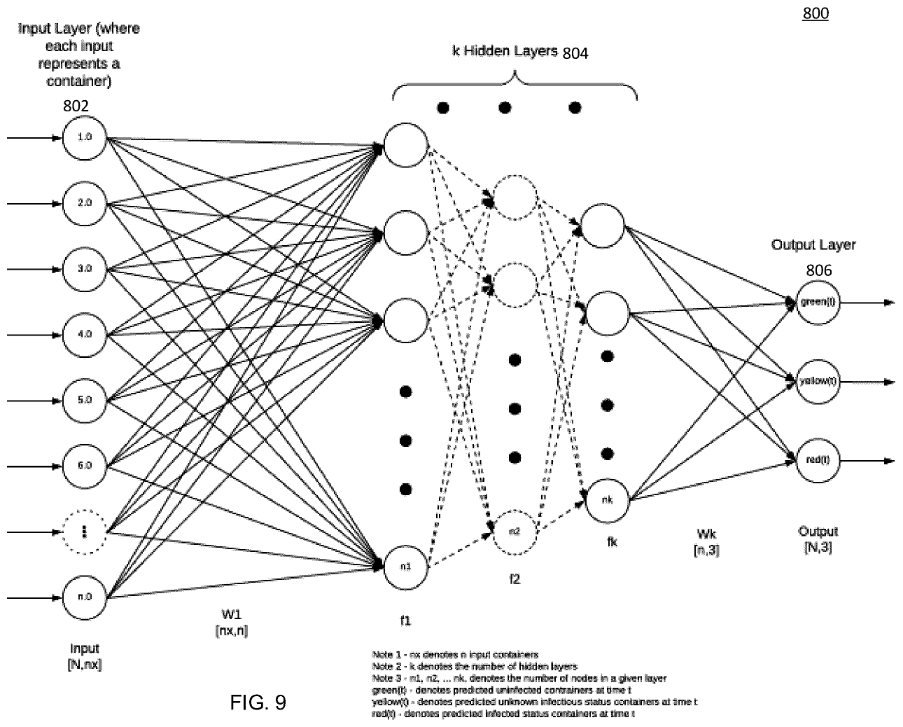

In the example of FIG. 9, neural network 800 may add an input layer 802, one or more hidden layers 804, and an output layer 806 for generating output results.

The input layer 802 receive inputs from one or more container. Each input 1-n to the input layer 802 represents a container 702a-702c. The first hidden layer 804 receives input from the input layer 802. Each subsequent hidden layer 804 receives inputs from a previous hidden layer. Each hidden layer 804 applies a set of weights to the inputs, and combines these weighted inputs to generate an output, which can in turn be provided as input to one or more neurons of a subsequent hidden layer. The outputs of the last hidden layer 804 are the input of output layer 806.

A hidden layer 804 uses filters to define the relationship between the outputs of the neurons of the previous layer and the outputs of the neurons of the current layer. A filter comprises a set of weights (also called parameters). In some neural networks, such as convolutional neural networks (CNNs), the weights of a filter are arranged into convolution kernels, such as 2D convolution kernels. Each kernel of a filter corresponding to a channel of the data input (i.e. an input activation map). The application of a single filter to the input volume (e.g. by convolving the kernels of the filter with the corresponding input activation maps of the data input) generates a single output activation map. The set of output activation maps generated by the set of filter of the convolution layer are the data output of the convolution layer.

For example, neural network 800 may be configured to provide time-based prediction output to identify specific containers, and hence specific physical assets associated with the containers in the real world, specific status (infected, not infected, unknown status) of the specific containers at specific time. These time-based predictions may be used to suggest mitigating actions for the containers to reduce the likelihood of further infection spread. For example, the system 10 may suggest that people in location x, y should start wearing masks at time t, and that buildings in the area around location x, y should start implementing new or more stringent cleaning protocols.

In order to infer infection risk and generate alert, the machine learning model 800 needs to be trained. In the example of a neural network, training a neural network involves learning or determining the appropriate weight values at different weight locations throughout the network. After being optimally trained to perform a given inference task, the weights of the neural network will not all contribute equally to the final inference outputs: some weights will have high value due to their high contribution, while other weights will have low value due to their low contribution. If the weights are not properly trained (e.g., high value weights are misplaced or miscalibrated by training), then the trained network will perform with less accuracy. In the system 10, the machine learning model can be trained by a suitable set of training data to determine appropriate weights.

The trained machine learning model can be used to create and apply models for performing inference tasks, such as inferring infection risk of a container and an outbreak or pandemic in a specific region.

The neural network 800 can be a CNN. The neural network 800 has been simplified, is not intended to be limiting and is provided for the purpose of illustration only. The input data may be sensory data from sensors 701 associated with containers 702a-702c.

In some examples, the neural network 800 may also include a preprocessing block for performing various operations (e.g., normalization) to prepare the input data for the neural network 800.

Certain adaptations and modifications of the described embodiments can be made. Therefore, the above discussed embodiments are considered to be illustrative and not restrictive.

What is claimed is:

1. A system for identifying infection risk, comprising:
one or more sensors associated with each of a plurality of physical assets for generating a signal when two or more physical assets of the plurality of physical assets are in a predetermined proximity; and
a server configured to receive the signal from the one or more sensors, and to identify an infection status of each of the two or more physical assets.

2. The system of claim 1, wherein when one of the two or more physical assets has an infected status, the server generates an alert to other physical assets of the two or more physical assets.

3. The system of claim 2, wherein the server set a status of the other physical assets as infected.

4. The system of claim 2, wherein the server set a status of each of the two or more physical assets as uninfected after each of the two or more physical assets has a disinfection transaction.

5. The system of claim 2, wherein the server set a status of each of the two or more physical assets as uninfected after each of the two or more physical assets has a disinfection transaction.

6. The system of claim 1, wherein the one or more sensors are Internet of Thing (IoT) sensors.

7. The system of claim 1, wherein the server defines each physical asset as a container and a digital twins in association with the one or more sensors.

8. The system of claim 7, wherein the container is an object container represent a physical asset or an action container representing an action applied to the object container.

9. The system of claim 7, wherein the container is a vehicle container, an individual container, or a building container.

10. The system of claim 1, wherein the server identifies the infection status using a machine learning model.

11. The system of claim 1, wherein the server is configured to record information from the signal in a blockchain.

12. The system of claim 1, wherein the collected information protects anonymity of individual persons participating within the system.

13. The system of claim 6, wherein the sensors are adapted to needs identified by a specific region, area, or jurisdiction.

14. The system of claim 1, wherein the physical assets include people.
